(19) United States
(12) Patent Application Publication
(10) Pub. No.: US 2026/0144438 A1
(43) Pub. Date: May 28, 2026

(54) EAR SPECULUM WITH FLUID DELIVERY DEVICE

(71) Applicant: The Feinstein Institute for Medical Research, Manhasset, NY (US)

(72) Inventor: Ali Ahmadizadeh, Riverdale, NY (US)

(21) Appl. No.: 19/262,375

(22) Filed: Jul. 8, 2025

Related U.S. Application Data
(60) Provisional application No. 63/725,286, filed on Nov. 26, 2024.

(51) Int. Cl. A61B 1/227; A61B 1/00; A61B 1/06; A61B 1/32; A61M 31/00

(52) U.S. Cl. CPC A61B 1/227; A61B 1/00094; A61B 1/0669; A61B 1/32; A61M 31/00; A61M 2210/0662

(57) ABSTRACT

A device for examining an ear canal and selectively applying treatment fluid to the ear canal is provided. The device includes a body and a reservoir. The body is double walled and includes an interior wall and an exterior wall separated by a gap. The interior wall is open at proximal and distal ends defines a viewing channel through the body so that the device functions as an ear speculum. A fluid channel is provided in the gap between the walls. A delivery aperture is provided at the distal end of the fluid channel. A reservoir holding the fluid is provided in the proximal portion of the fluid channel. To deliver the fluid, a practitioner applies pressure to the reservoir to displace the fluid along the fluid channel and out from the delivery aperture.

This application claims priority under 35 U.S.C. § 119 to U.S. Provisional Patent Application No. 63/725,286, filed on Nov. 26, 2024. The disclosure of that application is incorporated herein by reference.

### BACKGROUND

### Field

This disclosure relates to a device and method for delivering a fluid, and particularly, but not exclusively, to an apparatus and method for delivering fluid to an ear. In particular, the disclosure relates to a device to facilitate treatment of the ear with a treatment fluid while visualizing the ear canal.

Medical examination and treatment of body cavities is often provided using a speculum that allows a practitioner to visualize the inner surface of the cavity. The practitioner holds the proximal portion of the speculum and inserts the distal portion into the body cavity. A central opening through the speculum allows the practitioner to visualize the inner surface of the cavity.

To examine the outer eardrum and the inside surface of the ear canal, a practitioner typically uses an ear speculum. Known ear specula have a generally conical shape with a wider proximal end and a narrow distal tip. The tip is sized to fit into the ear canal. The central portion of the speculum is hollow, allowing the practitioner to look out through the distal tip to visualize the eardrum and surrounding tissues. The speculum may be combined with a light source, for example, by connecting the speculum to an otoscope.

A practitioner may need to deliver medications to the inner ear. For example, if a patient has a build-up of cerumen or an infection in the ear canal, the practitioner may need to deliver an antibiotic or a dissolving agent such as hydrogen peroxide to the site of the buildup or infection.

Conventionally, ear droppers are generally in the form of a squeezable container with an elongated nozzle and applicator tip that are shaped to reach into the ear canal. The container may be made of a flexible plastics and is arranged to be squeezed by a person's fingers. The nozzle is provided with a cover, such as a screw-on cap to close the applicator tip. Medication is contained in the plastic container. In use, the cap is removed and the ear dropper is maneuvered into position such that the applicator tip is partially inserted into the ear/ear canal so that the fluid can be delivered into the ear canal. Once the user has positioned the applicator in the correct position, the user squeezes the plastic container to force one or more drops of the medication out of the applicator and into the ear canal.

The application of fluid within the ear canal with such an ear dropper may be difficult, particularly with pediatric patients and often leads to medication not being applied in the correct location or falling onto surrounding surfaces causing a mess. Further, generally, the health care provider must first perform an exam using medical instruments, such as a speculum connected with an otoscope to diagnose the condition. To treat the condition, the provider removes the speculum and then reengages the ear canal with the dropper to deliver medications, such as an antibiotic. The practitioner may need to insert the speculum again to confirm that the medication was applied properly. This may increase the discomfort felt by the patient and may increase the time it takes for a health care provider to provide medical care. In addition, when the practitioner inserts the dropper, he or she cannot visualize where within the ear canal the medication is being delivered.

Thus, there is a need for a device that would allow a healthcare provider to apply a medication or cleaning solution via a single user dropper that also allows for visual confirmation of the appropriate application while performing an exam.

### SUMMARY

The present disclosure relates to a device for delivering fluid to the ear canal that overcomes these problems.

According to one aspect, there is provided a speculum and fluid delivery device that allows for a healthcare provider to visualize and deliver fluid to the ear canal simultaneously. The device eliminates the need for the healthcare provider to reinsert a separate instrument, such as a dropper bottle, to administer fluid to the ear canal thus reducing discomfort for the patient and time required for the healthcare provider.

According to one embodiment, the speculum and fluid delivery device has a body, a reservoir and a plunger. The body includes a distal region that is generally conical in shape and is sized to fit into the ear canal of a patient. According to one embodiment, the device includes a proximal region that is generally cylindrical in shape to provide a surface for a healthcare provider to hold the device. The body further includes an interior wall and an exterior wall. The walls are coupled by at least one coupler to define a fluid channel. The interior wall is hollow with an aperture at the proximal end, an aperture at the distal end that defines a viewing channel. The space between the interior wall and exterior wall define an actuator aperture at the proximal end, a dispensing aperture at the distal end. The fluid channel between the interior wall and exterior wall connects the two apertures. A reservoir holding a pre-determined amount of a treatment fluid is housed within the proximal end of the fluid channel. According to one embodiment, the reservoir is formed by a bladder. According to another embodiment, the reservoir is formed at the proximal portion of the fluid channel at least partially by the inner surface of the exterior wall and the outer surface of the interior wall.

According to one embodiment, a plunger is inserted through the actuator aperture. The plunger is generally cylindrical in shape and includes a proximal portion and a distal portion. The distal portion is sized and dimensioned so that at least a portion of the distal portion fits through the actuator aperture to contact the reservoir at the proximal portion of the fluid channel. The plunger may include one or more channels that are aligned with the couplers that join the interior and exterior walls. When the plunger is inserted into the actuator aperture, the channels align with respective ones of the couplers. According to one aspect, engagement of the channels with the couplers helps to align the plunger with the fluid channel. As the distal portion of the plunger is inserted into the fluid channel, the distal portion of the plunger engages the reservoir. Force applied by a practitioner onto the plunger causes fluid in the reservoir to be displaced. According to one embodiment, the reservoir includes a sealed bladder holding a selected treatment fluid. The bladder includes one or more weakened portions that rupture when sufficient force is applied to the plunger, releasing the fluid and displacing it through the fluid channel and out of the dispensing aperture at the distal end of the fluid delivery device.

According to one embodiment, the plunger has a central opening that aligns with the viewing channel. This allows a practitioner to look through the plunger and along the viewing channel to visualize the ear canal and, at the same time, apply force to the plunger to deliver the treatment fluid. According to one embodiment, the plunger acts as a syringe, allowing the practitioner to deliver a selected amount of fluid to the ear canal. This allows the practitioner to visualize the application of the fluid, to determine if sufficient fluid has been delivered, and to deliver additional fluid by further displacing the plunger.

According to another embodiment, the speculum and fluid delivery device is packaged as a single-use device. The device may be sterilized and hermetically sealed in a bag or other container that the practitioner opens at the time of treatment. This may have the advantage of quick and immediate use to treat a patient. The device may be disposed of, to avoid transferring infection from one patient to another.

According to another embodiment, the device has a body and a reservoir. The body includes a distal region that is generally conical in shape and a proximal region that is generally cylindrical in shape. The body further includes an interior wall and an exterior wall coupled to the interior wall by at least one coupler. The interior wall is hollow with apertures at the proximal and distal ends to define a viewing channel that connects the two apertures. As with previous embodiments, the space between the interior wall and exterior wall defines a fluid channel with a delivery aperture at the distal end. The proximal end of the fluid channel is closed and houses a reservoir. The reservoir holds a selected amount of a treatment fluid. According to one aspect, at least a portion of the exterior wall is deformable so that by squeezing the exterior wall, the reservoir is compressed to displace the fluid from the reservoir, along the fluid channel and out through the delivery aperture. According to one embodiment, the reservoir is formed by a bladder. The bladder includes one or more weakened portions. As force is applied to the deformable portion of the exterior wall, the exterior wall deforms and applies pressure to the reservoir causing one or more of the weakened portions to rupture to displace the fluid through the fluid channel and out of the delivery aperture at the distal end of the fluid delivery device.

According to one embodiment, the speculum and fluid delivery device has a body formed by an interior wall and an exterior wall separated by a gap to create an annular fluid channel. The channel is open at the distal end to form a delivery aperture. The body includes a distal region that is generally conical in shape and is sized to fit into the ear canal of a patient and a proximal region that is generally cylindrical in shape to provide a surface for a healthcare provider to hold the device. The walls are coupled at their proximal ends to form a fluid-tight seal between the interior and exterior walls. According to one embodiment, the seal is facilitated by an o-ring captured between proximal ends of the walls. The exterior wall includes an opening that extends into the fluid channel. A capsule formed from a deformable material extends through the opening. The capsule includes a weakened portion positioned in the fluid channel. The capsule holds a pre-determined amount of a treatment fluid. In use, a practitioner positions the distal end of the device in the ear canal or other bodily opening of a patient and to visualize tissue in the cavity. The practitioner applies force to the capsule, rupturing the weakened portion to deliver fluid through the fluid channel to the surface of the tissue.

According to a further embodiment, an outer surface of the interior wall includes two or more channel separators. The channel separators contact the inner surface of the exterior wall to define two or more channels that are each open at their distal ends and that are sealed at their proximal ends. The capsule extends into one of the channels to form the fluid delivery channel. Another of the channels is connected with a suction port that extends through the exterior wall to form a suction channel. The suction port may be connected with a source of vacuum, such as a hand-operated pump or hospital vacuum system. The vacuum source may include a valve that allows a practitioner to selectively apply suction via the suction channel to draw fluids and debris from the ear canal or other body cavity.

According to one embodiment, the interior wall defines a viewing channel along a longitudinal axis of the device. A magnifier is provided at the proximal end of the viewing channel. According to one embodiment, the magnifier is connected to the body of the device by a hinge. The hinge allows the magnifier to swing into alignment with the longitudinal axis so that a practitioner can see a magnified image of the surface being visualized (e.g., the ear drum or inner surface of the ear canal). When the practitioner does not wish to use magnification, the magnifier can be rotated away from the viewing channel. According to one embodiment, the magnifier includes a magnifying lens and a light source. The light source can be selectively activated to allow the practitioner to better visualize the body cavity while applying treatment fluid held in the capsule.

According to another embodiment, a kit or collection of ear specula are preloaded with a variety of treatment fluids for treating various conditions. The specula are each individually wrapped. In use, a healthcare provider selects a speculum that includes an appropriate fluid to treat a particular patient. The provider opens the package, uses the speculum to visualize the outer ear and to deliver the fluid. According to one embodiment, the speculum is for single use and the provider disposes of it, potentially reducing the risk of transmitting disease. According to another embodiment, the speculum is collected and refurbished.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a perspective view of a device for delivering treatment fluid while visualizing an ear canal according to an embodiment of the disclosure;

FIG. 2 is another perspective view of the device of FIG. 1;

FIG. 3 is a cross-section view of the device of FIG. 1;

FIG. 4 is a planar view of a proximal end of the device of FIG. 1;

FIG. 5 is a side perspective view of the device of FIG. 1 being inserted into an ear canal;

FIG. 6 is a side perspective view of the device of FIG. 1 delivering treatment fluid to the ear canal;

FIG. 7 is a perspective view of a device for delivering treatment fluid while visualizing an ear canal according to another embodiment of the disclosure;

FIG. 8 is another perspective view of the device of FIG. 7;

FIG. 9 is a cross-section view of the device of FIG. 7;

FIG. 10 is a planar view of a proximal end of the device of FIG. 7;

FIG. 11 is a side perspective view of the device of FIG. 7 being inserted into an ear canal;

FIG. 12 is a side perspective view of the device of FIG. 7 delivering treatment fluid;

FIG. 13 is a perspective view of a device according to embodiments of the disclosure being attached to a medical instrument;

FIG. 14A is a perspective view of a device for delivering treatment fluid while visualizing an ear canal using magnifier positioned along a viewing channel according to another embodiment of the disclosure;

FIG. 14B is a perspective view of the device of FIG. 14A with the magnifier positioned away from the viewing channel;

FIG. 15 is a exploded view of the device of FIG. 14A;

FIG. 16 is a cross-section view of the device of FIG. 14A;

FIG. 17A is a perspective view a magnifier according to embodiments of the disclosure

FIGS. 17B and 17C are exploded views of the device of FIG. 17A;

FIGS. 18A, 18B, and 18C are a perspective view, a cross-section view, and a proximal end view, respectively, of an exterior wall according to embodiments of the disclosure;

FIGS. 19A, 19B, and 19C are a perspective view, a cross-section view, and a proximal end view, respectively, of an interior wall according to embodiments of the disclosure; and

FIGS. 20A, 20B, 20C, and 20D are front and rear perspective views, a bottom view and a front view of a capsule according to embodiments of the disclosure.

### DETAILED DESCRIPTION

The present disclosure provides descriptions of embodiment for fluid delivery devices for delivery fluids to the ear, and more specifically, the ear canal. For ease of description, the terms “distal,” “distally,” “distal of” and the like will be used throughout this disclosure to refer to the direction or relative position away from the operator of the device and toward the body of a patient being treated using the device. The terms “proximal,” “proximally,” “proximal of” and the like will be used throughout this disclosure to refer to the direction toward the operator of the device and away from the body of a patient being treated using the device.

For ease of description, the user operating the device may also be referred to herein as the “healthcare provider,” “medical provider,” or practitioner.

While several embodiments are described, the new subject matter described in this patent disclosure is not limited to any one embodiment or combination of embodiments described herein, but instead encompasses numerous alternatives, modifications, and equivalents. In addition, while numerous specific details are set forth in the following description to provide a thorough understanding, some embodiments can be practiced without some or all such details. Moreover, for the purpose of clarity, certain technical matter that is known in the related art has not been described in detail in order to avoid unnecessarily obscuring the new subject matter described herein. It should be clear that individual features of one or several of the specific embodiments described herein can be used in combination with features of other described embodiments or with other features. Further, like reference numbers and designations in the various drawings indicate like elements.

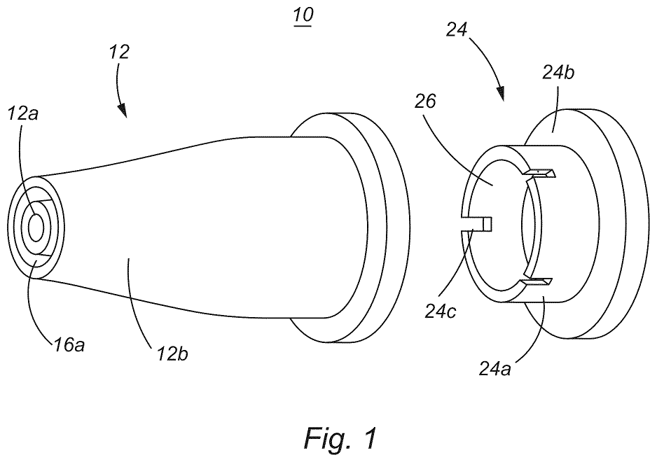

Referring to FIGS. 1-6, an exemplary embodiment of a speculum and fluid delivery device 10 for delivering fluid to an ear canal is shown. According to one embodiment, the fluid delivery device shown in FIGS. 1-6 allows a practitioner to visually inspect a patient's ear canal, for example, during a medical examination. The provider may determine a medication or cleansing fluid is required. Device 10 allows the provider to visually confirm that the fluid is being delivered to the desired area.

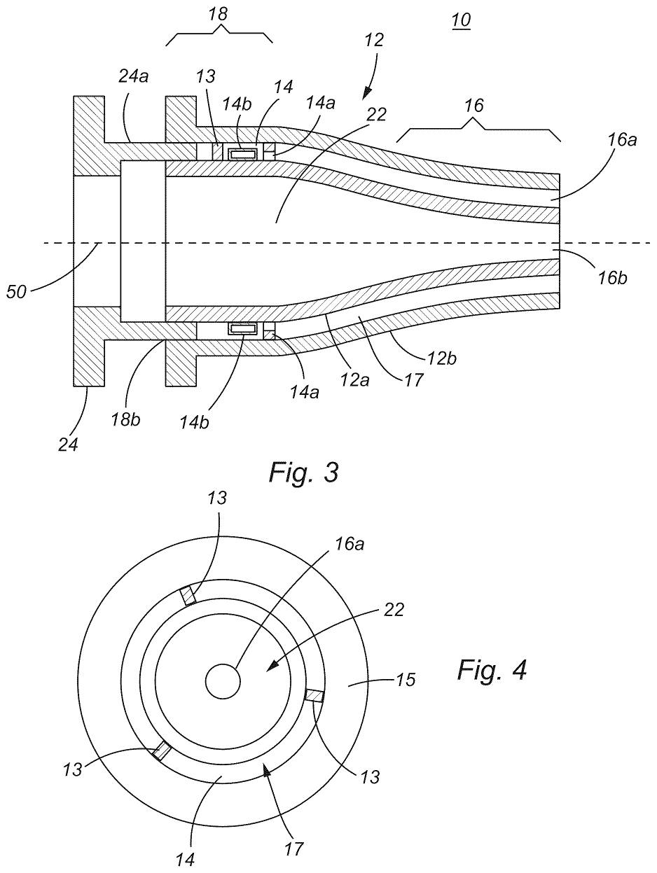

As shown in FIGS. 1-4, device 10 includes a body 12, a reservoir 14 and a plunger 24. In one embodiment, body 12 may be made from plastic, metal, glass, or a combination thereof. In a preferred embodiment, device 10 is disposable and body 12 is made of plastic. According to another preferred embodiment, body 12 is formed from a medically suitable, moldable polymer. As shown in the cross-section view on FIG. 3, body 12 is double walled with reservoir 14 positioned between the walls at the proximal region 18 of body 12. One or more couplers 13 may be provided between the interior wall 12a and the exterior wall 12b to hold the wall apart to define a gap between the walls.

Couplers 13 may be integrally formed with the interior wall 12a and the exterior wall 12b, for example, by injection molding, or may be coupled together via welds, adhesives or other known methods. In the exemplary embodiment shown, the interior wall 12a and exterior wall 12b are joined by three couplers 13. However, more or fewer couplers 13 may be used.

Interior wall 12a is hollow and open at both ends to provide viewing channel 50 along the longitudinal axis of device 10. One or more dispensing apertures 16a are provided at the distal end of body 12. According to one embodiment, dispensing aperture 16a is annular and is formed by the distal end of the gap between walls 12a, 12b. According to another embodiment, walls 12a and 12b are connected with one another at the distal end of body 12 and one or more dispensing apertures 16a are provided through the connected walls.

Distal portion 24a of plunger 24 is shaped to fit into the gap between walls 12a, 12b. Slots 24c may be provided to align with respective couplers 13 so that, when distal portion of the plunger is inserted into the gap, couplers 13 slide along slots 24c. According to one embodiment, this helps guide the plunger into the gap between the walls, as will be described below. According to one embodiment, plunger 24 is annular with an open central region to provide a unobstructed view along viewing channel 50.

FIG. 3 shows a cross-sectional view of the delivery device 10. In the exemplary embodiment shown, the interior wall 12a and exterior wall 12b separated by a gap that forms a fluid channel 17 therebetween. According to one embodiment, fluid channel 17 is annular and is formed by the inner surface of exterior wall 12b and interior wall 12a.

Reservoir 14 is formed in the gap between walls 12a, 12b within proximal portion 18. According to one embodiment, a reservoir wall 14a is provided between walls 12a and 12b to form a closed space within proximal region 18 to create reservoir 14. According to one embodiment, reservoir wall 14a includes one or more openings in fluid communication with fluid channel 17. As shown in FIG. 3, when plunger 24 is inserted between walls 12a and 12b, distal portion 24a of plunger 24 moves into the closed space forming reservoir 14. Treatment fluid provided in reservoir 14 is displaced when force is applied to the plunger 24, causing the fluid to flow through openings in reservoir wall 14a, along fluid channel 17, and out through dispensing aperture 16a.

According to one embodiment, bladder 14b may be provided in reservoir 14. Bladder 14b is filled with a selected amount of treatment fluid. Displacement of plunger 24 into proximal portion 18 causes bladder 14b to rupture to release the fluid and to displace the fluid along fluid channel 17 and out through dispensing apertures 16a.

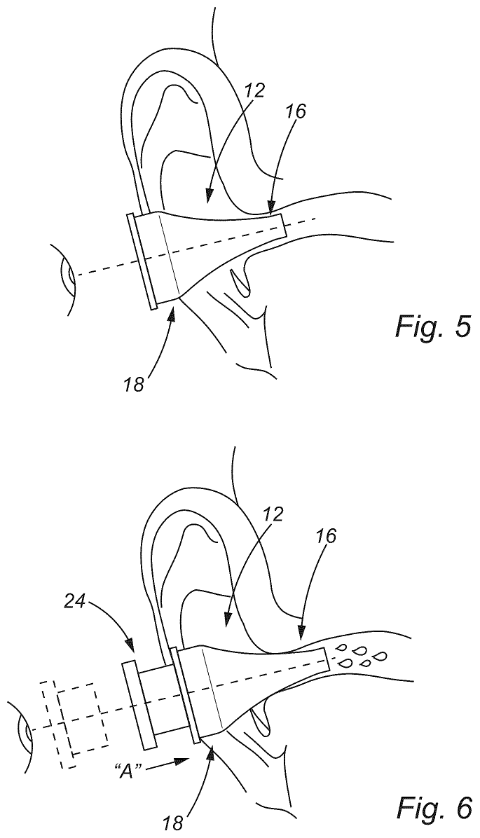

According to one embodiment, bladder 14b is formed from a flexible material, such as silicone, rubber, plastic or any other suitable material and configured to hold a pre-determined amount of fluid. The fluid may be any suitable ear medicament fluid or any other fluid suitable for use with an ear, for example, medicine, cleaning solution, saline solution or any other such fluid adapted for use in treating conditions of the ear. In an exemplary embodiment, bladder 14b at least partially ruptures as a result of being deformed under pressure from plunger 24. For example, the bladder may have a weakened distal side wall and as pressure is put on the proximal side of the bladder, at least a portion of the weakened distal side wall ruptures so that the fluid travels through channel 17 and is expelled through aperture 16a as shown in FIG. 6. In one exemplary embodiment, reservoir 14 is integral with the body 12. In another exemplary embodiment, reservoir 14 is configured to couple with the interior wall 12a and exterior wall 12b within at least a portion of the proximal region 18 of fluid channel 17 via adhesives, welds, or friction fit.

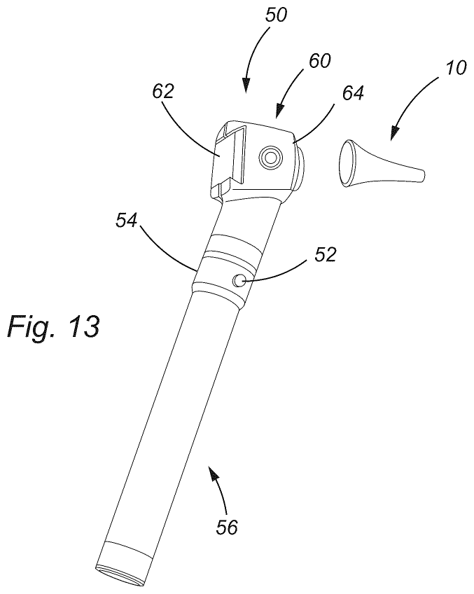

According to some embodiments, proximal region 18 includes features so that device 10 can be releasably attached to an instrument, such as an otoscope, such as shown in FIG. 13. Such attachment features may include mechanical attachment features, such as a dovetail connection, a bayonet connection, a threaded connection, a snap-fitting mount or other suitable arrangements for releasable connecting device 10 to an otoscope.

Distal region 16 tapers to a size suitable for insertion into a patient's ear canal. According to one embodiment, the outer diameter of distal portion 16 is between about 3 millimeters and about 8 mm. According to one embodiment, device 10 may be provided as a kit wherein devices with a variety of tapered distal portions 16 are sized for different patients (e.g., adult, pediatric, infant), or for examination of body orifices other than the ear.

Continuing to refer to FIG. 3, in an exemplary embodiment, viewing channel 22 is defined by interior wall 12a and is in communication with aperture 18a of the proximal region 18 and aperture 16b of the distal region 16. The viewing channel 22 is configured such that when the distal region 16 is inserted into an ear canal, a healthcare provider is able to look through viewing channel 22 along viewing axis 50 to visually inspect the ear canal, visually confirm the proper placement of fluid within the ear and/or visually determine the amount of fluid being delivered, i.e., the number of drops of fluid being administered, as shown in FIG. 6.

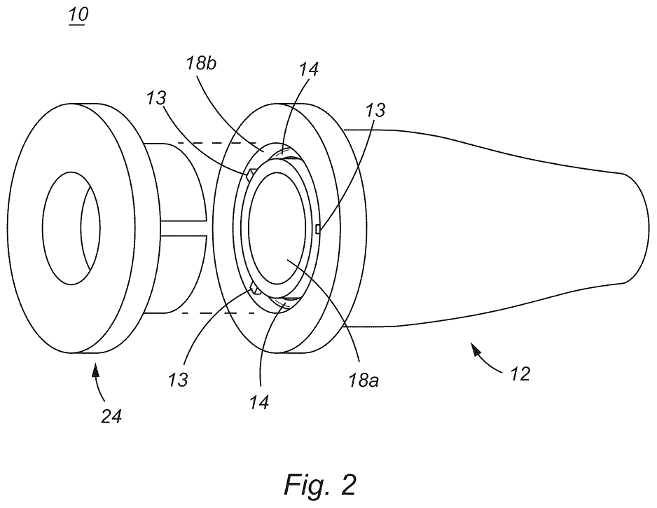

Referring to FIGS. 1-2, the plunger 24 is generally cylindrical in shape and formed from a rigid material, such as plastic, glass, metal or any other suitable material. The plunger 24 includes a distal portion 24a coupled with a proximal portion 24b with a viewing channel 26 therethrough. In one embodiment, the distal portion 24a is monolithically formed with the proximal portion 24b. In another embodiment, the distal portion 24a and proximal portion 24b are joined via welds, adhesives, or fasteners. The distal portion 24a is configured to fit into actuator opening 18b and partially along a portion of the proximal region 18 of fluid channel 17 to displace fluid in reservoir 14. Plunger 24 includes grooves or channels 25 that are arranged to correspond with respective ones of couplers 13. The at least one groove or channel 25 may assist with the alignment of distal portion 24a with the fluid channel 17. As the distal portion 24a is inserted into fluid channel 17, the distal portion 24a engages with reservoir 14. Pressure applied by the healthcare provider squeezes reservoir 14 to induce fluid to flow through fluid channel 17 and be expelled out of apertures 16a as shown in FIG. 6. In the exemplary embodiment shown, the proximal portion 24b is generally larger in size than the distal portion 24a so that the plunger 24 is easier to grasp and manipulate for the healthcare provider. However, it is contemplated that the proximal portion 24b may be generally the same size as the distal portion 24a or smaller in size than the distal portion 24a. According to one embodiment, plunger 24 is provided as a separate component from body 12. According to another embodiment, distal portion 24a is captured within proximal portion 18, for example, by a snap-fit connection and so that distal portion 24a is slidable along channel 17 to displace fluid from reservoir 14.

Referring to FIGS. 5 and 6, an exemplary embodiment for the use of fluid delivery device 10 of FIG. 1 is described. Initially, the health care provider inserts the distal region 16 at least partially into the ear canal of a patient. The health care provider is then able to look through the viewing channel 22 to visually assess the condition of the patient's outer ear to determine the appropriate intervention or if intervention is necessary. Upon the determination that medical intervention is necessary, for example the patient needs antibiotics applied to the ear canal, the health care provider inserts the distal portion 24a of the plunger 24 into proximal region 18 of channel 17 such that viewing channel 22 and viewing channel 26 are aligned. This allows the healthcare provider to visualize the tissue to be treated and confirm the fluid is applied to the desired location. The healthcare provider then applies a force to press the distal portion 24a of plunger 24 into reservoir 14 in the direction of arrow “A,” as shown in FIG. 6. According to one embodiment, this increases the pressure in the reservoir 14 displacing fluid through openings in reservoir wall 14a, through channel 17, out of apertures 16a, and onto the surface being treated by the provider.

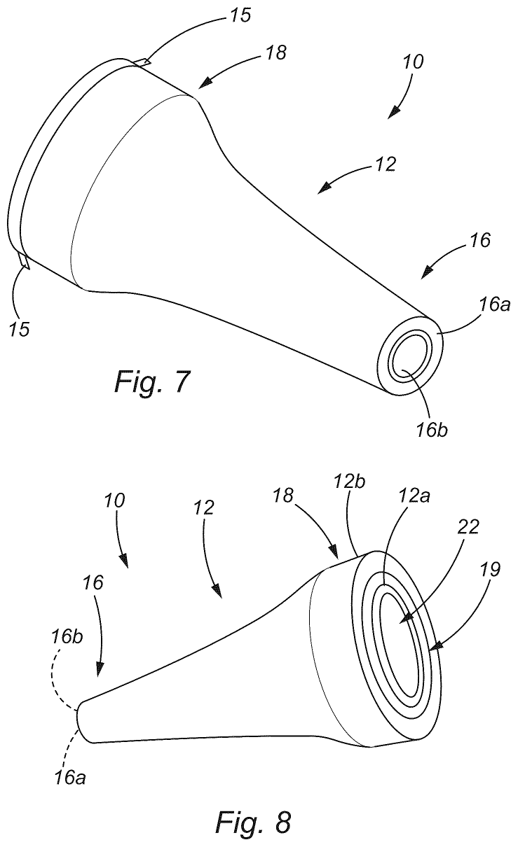

Referring to FIGS. 7-12, further exemplary embodiments of a speculum and fluid delivery device 10 for delivering fluid to an ear canal according to the present disclosure are shown. In these exemplary embodiments, device 10 provides proximal and distal openings 18b, 16b, and viewing channel 22 to allow a healthcare provider to visualize a patient's ear canal, as described in the previous embodiments.

Referring now to FIGS. 7-10, another exemplary embodiment of a speculum and fluid delivery device 10 is shown. As with previous embodiments, body 12 is double walled and includes an interior wall 12a coupled to an exterior wall 12b separated by fluid channel 17. According to one embodiment, interior wall 12a is connected with exterior wall 12b by closure 19. Closure 19 is an annular ring extending along facing surfaces of the interior and exterior walls 12a, 12b to define a closed proximal end of fluid channel 17. Interior wall 12a is separated from the inner surface of exterior wall 12b to define one or more delivery apertures 16a at a distal end of fluid channel 17.

According to one embodiment, interior wall 12a is formed from a relatively rigid material, such as a hard, molded plastic. At least a portion of the proximal region 18 of the exterior wall 12b is formed from a relatively flexible material, such an elastomer. This arrangement allows a practitioner to compress a portion of exterior wall 12b against interior wall 12a.

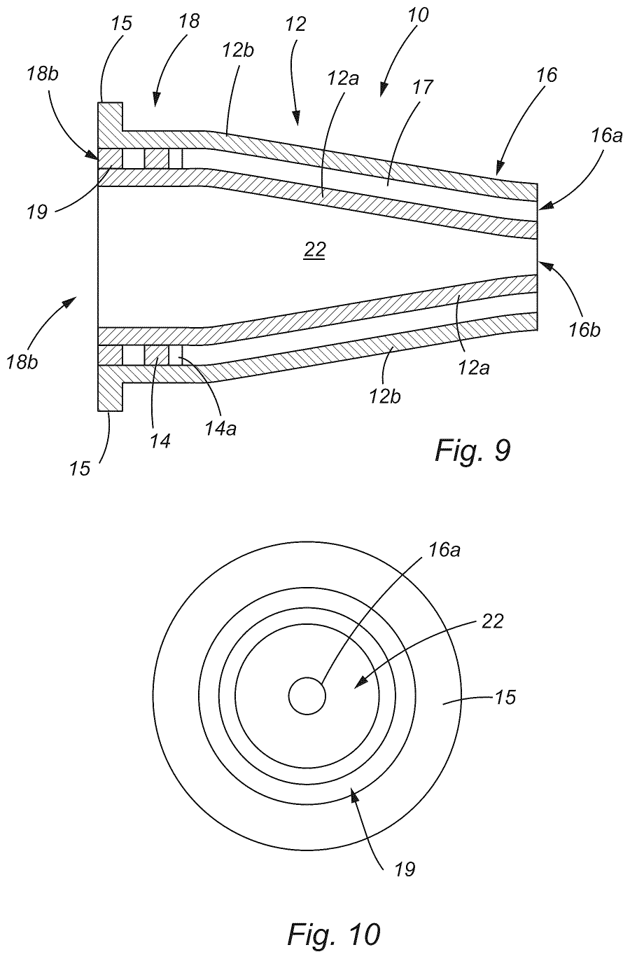

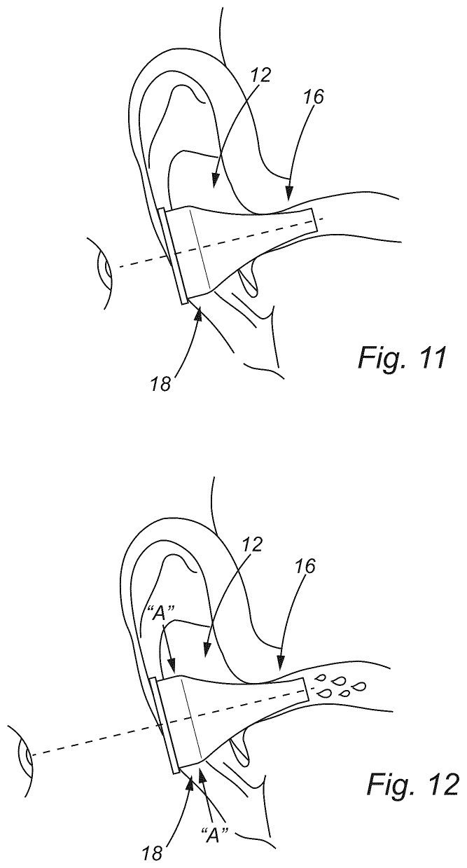

FIG. 9 shows a cross-sectional view of device 10. In the exemplary embodiment shown, the interior wall 12a and exterior wall 12b define a fluid channel 17 therebetween. Closure 19 at the proximal end of walls 12a and 12b closes the proximal end of fluid channel 17. Reservoir 14 is housed within the proximal portion of fluid channel 17. According to one embodiment, reservoir 14 is formed by the cavity distal of closure 19 at the proximal end of fluid channel 17. According to another embodiment, reservoir 14 is formed as a bladder filled with a selected treatment fluid. According to one embodiment, reservoir 14 includes a breakable seal 14a. When pressure is exerted in the direction of arrow “A” shown in FIG. 12, the exterior wall 12b deforms and applies pressure to the reservoir 14 rupturing seal 14a and displacing fluid contained within the reservoir 14 to flow through fluid channel 17 and to be expelled out of apertures 16a.

According to another embodiment, reservoir 14 is configured to hold a pre-determined amount of fluid, such as a treatment fluid. The fluid may be any suitable ear medicament fluid or any other fluid suitable for use with an ear, for example, medicine, cleaning solution, saline solution or any other such fluid adapted for use with an ear. In an exemplary embodiment, reservoir 14 is formed as a bladder filled with such a fluid and having a weakened portion that at least partially ruptures when pressure is applied to the bladder. For example, the bladder may have a weakened distal side wall and as pressure is placed on the bladder in the direction of arrow “A” shown in FIG. 12, at least a portion of the weakened distal side wall ruptures so that the fluid travels through fluid channel 17 and is expelled through aperture 16a as shown in FIG. 12. In one exemplary embodiment, reservoir 14 is integral with the body 12. In another exemplary embodiment, reservoir 14 is configured to couple with the interior wall 12a and exterior wall 12b within at least a portion of the proximal region 18 of fluid channel 17 via adhesives, welds, or friction fit.

Continuing to refer to FIG. 9, in an exemplary embodiment, a viewing channel 22 is defined by the interior wall 12a and is in communication with aperture 18b of the proximal region 18 and aperture 16b of the distal region 16. The viewing channel 22 is configured such that when the distal region 16 is inserted into an ear canal, a healthcare provider is able to look through the viewing channel 22 to visually inspect the ear canal, visually confirm the proper placement of fluid within the ear and/or visually determine the amount of fluid being delivered, i.e., the number of drops of fluid being administered, as shown in FIG. 12.

It is also contemplated within this disclosure that proximal region 18 may be releasably attached to a variety of otoscopes known in the art in a variety of ways known in the art, as shown in FIG. 13. For example, proximal region 18 may include mechanical attachment features, such as a dovetail connection, a bayonet connection, a threaded connection, a snap-fitting mount or other suitable arrangements for releasable connecting the device 10 to an otoscope.

Referring to FIGS. 11 and 12, an exemplary embodiment for the use of device 10 of FIGS. 7 and 8 is described. Initially, the health care provider inserts the distal region 16 at least partially into the ear canal of a patient. The health care provider is then able to look through the viewing channel 22 to visually assess the condition of the patient's outer ear to determine the appropriate intervention or if intervention is necessary. Upon the determination that medical intervention is necessary, for example the patient needs antibiotics applied to the ear canal, the health care provider applies a force with his or her fingers, i.e., squeezes, the flexible portion of the exterior wall 12b in the direction of arrow “A,” as shown in FIG. 12, and increases the pressure in the reservoir 14 causing a partial rupture of the bladder and/or rupturing seal 14a. The fluid held in reservoir 14 is displaced through fluid channel 17 and flows out of one or more apertures 16a and into the ear canal of the patient. According to one embodiment, the reservoir is filled with a pre-determined amount of the fluid such that a single dosage is provided when reservoir 14 is displaced.

An otoscope is a well-known hand-held medical examination tool used to examine body cavities. The otoscope may include a light source and magnifying lenses.

FIG. 13 illustrates an otoscope 50 for use with a speculum and fluid delivery device 10 according to embodiments of the disclosure to allow a healthcare provider to illuminate and visualize the ear canal of the patient. According to embodiments of the disclosure, device 10 is adapted to connect with otoscope 50 to introduce fluid, such as ear medicament or cleaning solution, into the ear canal without having to withdraw from the ear canal and reinsert with a separate device to provide treatment. This may reduce the level of discomfort for a patient and speed up the examination time for the busy healthcare provider.

The otoscope 50 includes a head 60, trigger 52, rotating member 54, and body 56. The head 60 includes a viewing window 62, attachment assembly 64, and a light. The light is contained within the head 60 and is therefore not shown in FIG. 13.

The attachment assembly 64 allows for specula to couple to the otoscope head 60. In this figure, fluid delivery device 10 is shown coupling to the attachment assembly 64 in place of a known speculum. The attachment assembly 64 may include mechanical features, such as a dovetail connection, a bayonet connection, a threaded connection, a snap-fitting mount or other suitable arrangements for releasably connecting the fluid delivery device 10 to the otoscope head 60.

In some instances, the viewing window 62 includes a magnification lens to magnify the view at distal region 16. In one embodiment, the magnification is a fixed magnification. In another embodiment, the magnification may be adjustable at the head 60.

Trigger 52 turns on and off the light within the head 60 which is used to illuminate the view from the distal region 16. Distal region 16 focuses the light from the otoscope head 60 into the orifice or opening 16b to illuminate adjacent tissues. In some instances, the rotating member 54 adjusts the intensity or the brightness of the light.

The viewing window 62 therefore provides a magnified and illuminated view of the tissue adjacent to opening 16b. In this manner, the healthcare provider is able to examine the tissues and determine if treatment using fluid in reservoir 14 is advisable. If so, the provider squeezes the proximal portion of device 10, rupturing seal 14a and delivering the predetermined amount of fluid through fluid channel 14 and out form apertures 16a to treat the adjacent tissues.

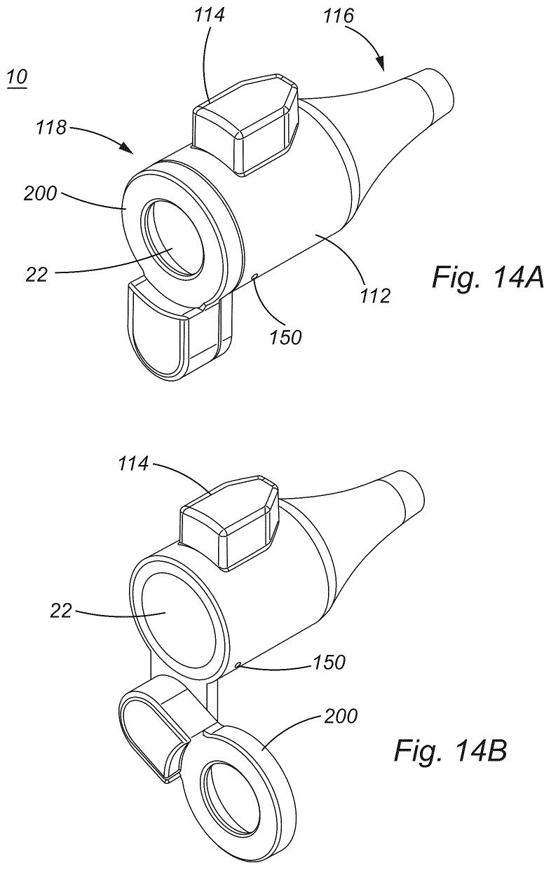

FIGS. 14A to 20D show device 10 according to a further embodiment of the disclosure. FIGS. 14A and 14B show perspective views of device 10, with and without magnifier 200 aligned with viewing channel 22. As shown in FIG. 14A, device 10 is formed by body 112 having a distal conical portion 116 for insertion into the ear canal or other body cavity. Proximal portion 118 is shaped to be grasped by a practitioner using the device to visualize and treat the bodily cavity.

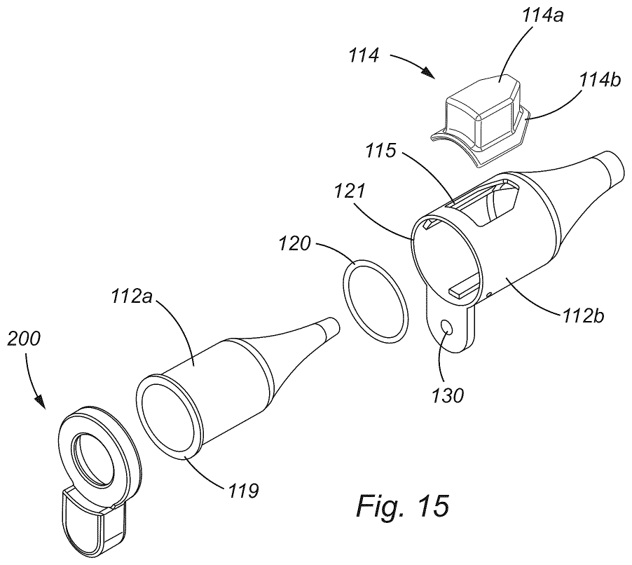

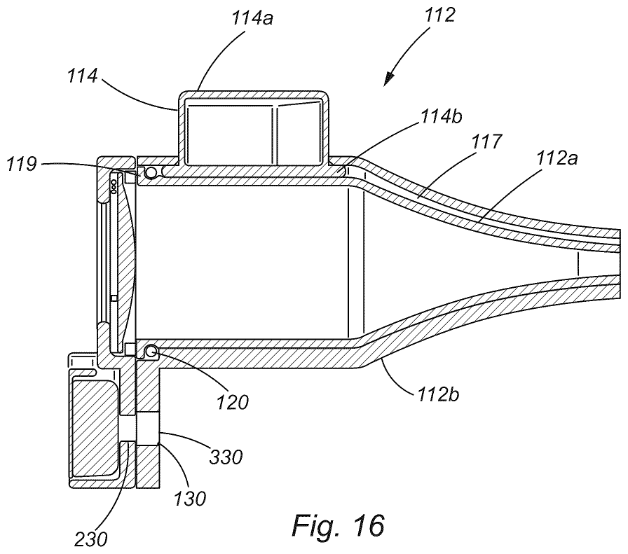

FIG. 15 shows an exploded view of the components of device 10 and FIG. 16 shows a cross section of the assembled device. Interior wall 112a is fitted concentrically inside exterior wall 112b. Walls 112a, 112b are separated by a gap forming one or more channels 117.

Interior wall 112a includes lip 119 at its proximal end. Exterior wall 112b includes a shoulder 121 at its proximal end. Lip 119 is shaped to abut with shoulder 121 and close the proximal end of the gap. According to one embodiment, lip 119 forms a snap fit with portions of exterior wall 112b. According to another embodiment, lip 119 is joined with exterior wall 112b using adhesives, welding, including ultrasonic welding, by co-molding, or by other techniques known to those of skill in the field of the disclosure. Lip 119 forms a seal with shoulder 121. According to one embodiment, o-ring 120 is provided between lip 119 and shoulder 121 to seal the proximal end of the gap.

As shown in FIG. 15, exterior wall 112b includes opening 115. Opening 115 is sized to receive capsule 114. Exterior portion 114a of capsule 114 extends outward from exterior wall 112b. Flange 114b of capsule 114 is larger than opening 115. As shown in the cross-section view in FIG. 16, when capsule 114 is positioned in opening 115 and interior wall 112a is secured within exterior wall 112b, flange 114b is captured between walls 112a, 112b.

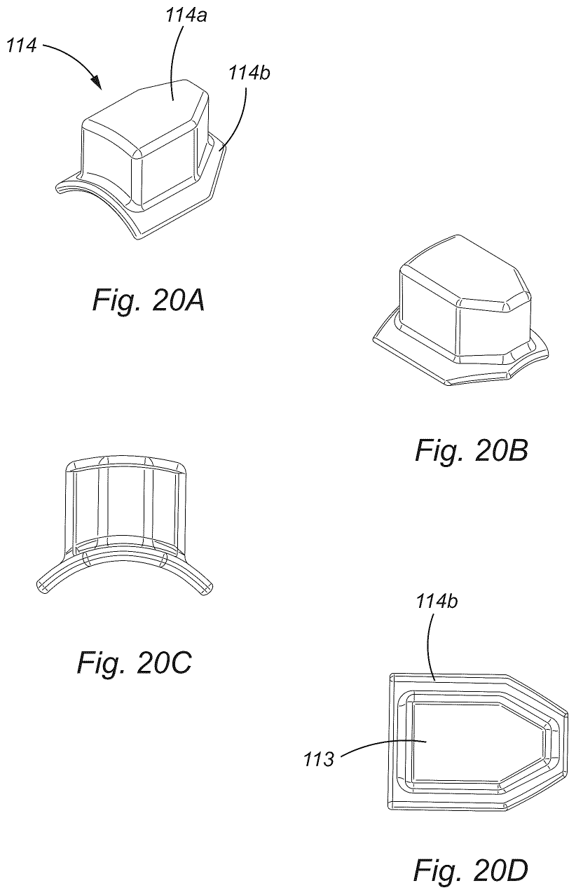

FIGS. 20A-20D show detailed views of capsule 114. Capsule 114 encloses a volume filled with a selected amount of treatment fluid, such as any of the treatment fluids described with respect to previous embodiments of the disclosure. According to one embodiment, the volume is between 0.5 cubic centimeters (cc) and about 5 cc. According to a preferred embodiment, the volume is about 1 cc.

As shown in FIG. 20D, rupturable portion 113 is provided on the side of capsule adjacent to flange 114b. As shown in FIG. 16, rupturable portion 113 is positioned within fluid channel 117. According to one embodiment, portion 113 is a panel supported by flange 114b. According to another embodiment, rupturable portion 113 is formed as part of flange 114b.

At least part of exterior portion 114a is formed from a deformable material, such as an elastomeric polymer or a metallic foil so that, when force is applied to the exterior portion, hydrostatic pressure of the fluid increases, rupturing portion 113 and releasing the treatment fluid into fluid channel 117.

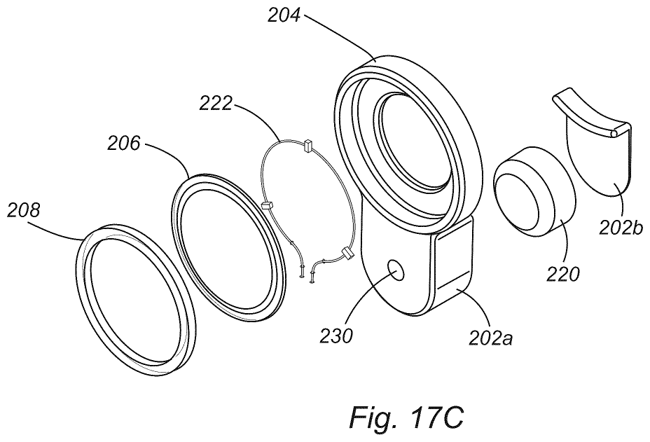

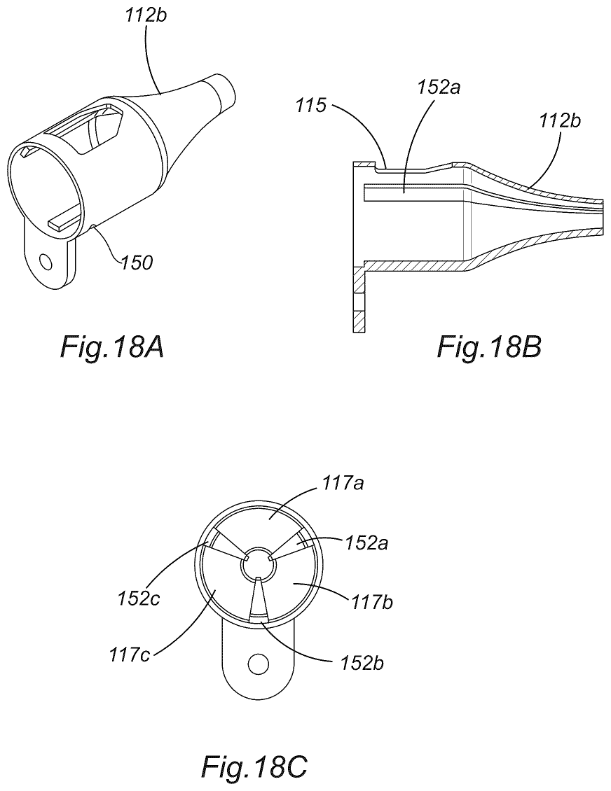

FIGS. 18A, 18B, and 18C show a perspective view, a cross-section view, and an elevation view, respectively, of exterior wall 112b. Wall 112b includes two or more channel separators 152a, 152b, 152c arranged radially and extending longitudinally along its inner surface. Separators 152a, 152b, 152c are sized to contact the outside surface of interior wall 12a to form separate channels. In the embodiment shown, three separators 152a, 152b, 152c form three channels 117a, 117b, 117c along the length of the gap between walls 112a, 112b. Three separators forming three channels are shown in this embodiment, but a lesser or greater number of separators could be provided within the scope of the disclosure.

Channel 117a is aligned with opening 115 to form a fluid delivery channel that receives fluid from capsule 114 when portion 116 is ruptured. As discussed with respect to previous embodiments, the practitioner can visualize the inside of the body cavity and, if necessary, apply force to capsule 114 to deliver treatment fluid.

As shown in FIGS. 14A and 14B, body 112 includes vacuum port 150. Vacuum port 150 is aligned with channel 117b to form a suction channel. According to one embodiment, port 150 is connected with a source of vacuum. The vacuum source may include a valve to allow a practitioner to selectively apply vacuum to port 150 to suction debris and fluids from the ear canal or other body cavity along suction channel 117b.

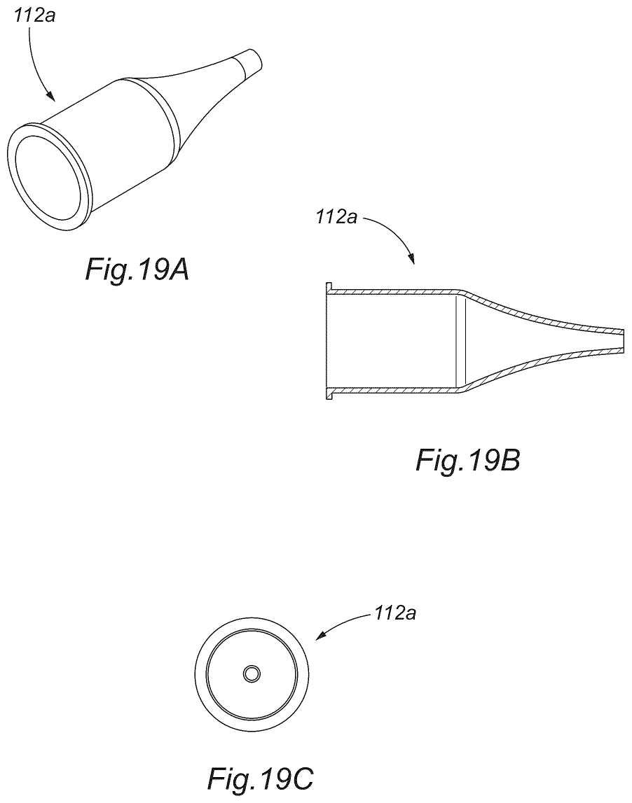

FIGS. 19A, 19B, and 19C show a perspective view, a cross-section view, and an elevation view, respectively, of interior wall 112a. According to one embodiment, the outer surface of wall 112a is smooth. Separators 152a, 152b, 152c on exterior wall 112b are shaped to conform to the surface of wall 112a so that the separators form fluid-tight barriers between channels 117a, 117b, 117c. According to another embodiment, instead of providing channel separators on exterior wall 112b, the separators are applied to the outside surface of interior wall 112a and extend outward to contact and form a fluid-tight connection with the inner side of exterior wall 112b.

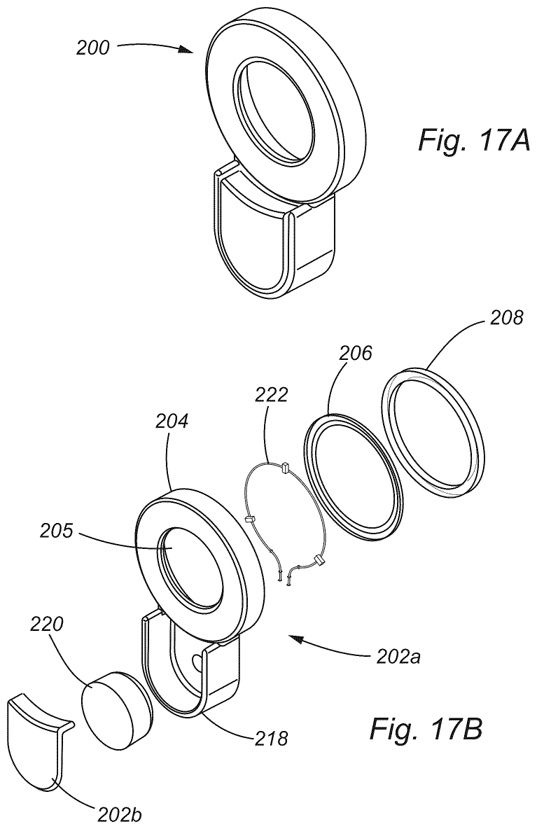

FIG. 17A shows a perspective view of magnifier 200 according to an embodiment of the disclosure. FIGS. 17B and 17C show exploded views of magnifier 200 from the proximal and distal directions, respectively. Housing 202a includes battery compartment 218 sized to hold battery 220. Battery cover 202b removably connects with housing 202a to enclose battery 220 and to allow a user to open compartment 218 to replace the battery.

Housing 202a includes annular portion 204 defining viewing opening 205. Lens 206 is fitted within annular portion 204 and aligns with opening 205. Lens 206 is held in place by bezel 208. According to one embodiment, bezel 208 and annular portion 204 include features to form a snap fit to secure lens 206 with housing 202a. According to another embodiment, bezel 208 is joined with housing 202a by adhesives, welding, or other techniques know to those of skill in the field of the disclosure.

According to one embodiment, light source 222 is positioned within annular portion 204 and arranged to illuminate region distal of magnifier 200. Light source 222 may be one or more light emitting diodes (LEDS) arranged radially along the periphery of lens 206. Electronic components including an on/off switch and/or brightness adjustment (not shown) may be provided within housing 202a, 202b to allow the practitioner to selectively deliver power from battery 220 to light source 222 to illuminate tissues being visualized during a procedure.

Magnifier 200 includes hinge opening 230. As shown in FIGS. 15 and 16, hinge opening 230 aligns with hinge opening 130 of body 112. As can be seen in FIG. 16, hinge pin 330 extends through hinge opening 203 and hinge opening 130 to rotatably connect magnifier 200 with body 112. As shown in FIG. 14A, magnifier 200 can be positioned so that opening 205 aligns with viewing channel 22 to provide a magnified view tissues along the ear canal or other body cavity distal of device 12. As shown in FIG. 14B, when magnification is not necessary, magnifier 200 is rotated away from viewing channel 22.

According to another embodiment, a kit or collection of devices 10 are provided. The collection includes devices with selected dimensions for examining different patients, for example, infant, pediatric, and adult sized instruments may be provided. According to further embodiments, the collection includes devices 10 that are each preloaded with a variety of different treatment fluids for treating various conditions. According to a further embodiment, the devices 10 in the collection are each individually wrapped, sterilized, and sealed. In use, a healthcare provider selects a device 10 from the collection with the appropriate size and loaded with a treatment fluid likely to be needed to treat a patient. The provider opens the wrapping, uses the device to visualize the outer ear and, if needed, actuates device 10 to deliver the fluid. According to one embodiment, the device is for single use and the provider disposes of it, potentially reducing the risk of transmitting disease from patient to patient. According to another embodiment, after it is used, the device is collected and refurbished.

While illustrative embodiments of the disclosure have been described and illustrated above, it should be understood that these are exemplary of the disclosure and are not to be considered as limiting. Additions, deletions, substitutions, and other modifications can be made without departing from the spirit or scope of the disclosure. Accordingly, the disclosure is not to be considered as limited by the foregoing description.

I claim:

1. A device for facilitating examination and delivery of treatment fluid into an ear canal, comprising:
a speculum body including an exterior wall and an interior wall, wherein the interior and exterior walls are separated by a gap to form a fluid channel, wherein an interior portion of the interior wall defines a viewing channel, wherein one or more delivery openings are provided at a distal end of the fluid channel; and
a reservoir adapted to hold the treatment fluid in fluid communication with the fluid channel, wherein a force applied to the reservoir displaces the fluid along the fluid channel and out through the delivery openings.

2. The device of claim 1, wherein the reservoir comprises a deformable portion adapted to be deformed by application of a force and a weakened portion in fluid communication with the fluid channel, wherein the force increases hydrostatic pressure in the reservoir to rupture the weakened portion and deliver the fluid along the fluid channel to the delivery opening.

3. The device of claim 2, wherein the deformable portion extends outward from the speculum body.

4. The device of claim 1, wherein interior wall is concentric with the exterior wall so that the gap is a substantially annular space, and wherein the fluid channel comprises at least a portion of the annular space between the interior and exterior walls.

5. The device of claim 4, wherein fluid channel comprises substantially all of the annular space.

6. The device of claim 4, further comprising one or more channel separators disposed in the annular space, wherein the two or more channel separators define two or more channels.

7. The device of claim 6, wherein the two or more channel separators comprise at least two channel separators, wherein the channel separators divide the annular space to define the fluid channel and at least one other channel.

8. The device of claim 7, wherein the device further comprises a vacuum port in fluid communication with the at least one other channel to form a suction channel.

9. The device of claim 4, wherein one of the interior wall and the exterior wall comprises a lip at a proximal end, wherein the lip contacts another of the interior and exterior wall to close the annular space at a proximal end of the body.

10. The device of claim 9, further comprising an o-ring positioned between the lip and the other of the interior or exterior wall to form a fluid-tight seal at the proximal end of the annular space.

11. The device of claim 1, further comprising a magnifier at a proximal end of the speculum body and adapted to be aligned with the viewing channel.

12. The device of claim 11, wherein the magnifier is connected with the speculum body by a rotatable hinge, wherein, in a first position, the magnifier is aligned with the viewing channel and, in a second position, the magnifier is positioned away from the viewing channel.

13. The device of claim 11, wherein the magnifier comprises a lens and a light source.

14. The device of claim 1, wherein the speculum body is adapted to connect with an otoscope.

15. A device for facilitating the delivery of a fluid into a body cavity, comprising:
a speculum body having a distal region that is generally conical in shape, the body including an exterior wall, an interior wall, and at least one channel separator between the exterior wall and interior wall, wherein the interior wall defines a viewing channel, wherein the interior and exterior wall are separated by a gap, wherein the gap and the channel separator define a fluid channel, and wherein the fluid channel is closed at a proximal end and extends to a distal end of the body to form a dispensing aperture; and
a capsule holding the fluid, wherein the capsule comprises a rupturable portion disposed between the walls and in fluid communication with the fluid channel and a deformable portion extending outward from the exterior wall, wherein a force applied to the rupturable portion displaces the fluid through the rupturable portion, along the fluid channel and out from the dispensing aperture.

16. The device of claim 15, comprising two or more channel separators, wherein the gap is divided into at least one fluid channel and at least one other channel.

17. The device of claim 16, further comprising a vacuum port in fluid communication with the at least one other channel to form a suction channel.

18. The device of claim 17, further comprising a vacuum source adapted to selectively apply suction through the vacuum port.

19. A method of using the device according to claim 15, comprising the steps of inserting the distal end of the body into a body cavity, visualizing tissue within the cavity through the viewing channel and applying the force to the deformable portion to deliver the fluid to the tissue through the dispensing aperture.
